(19) United States
(12) Patent Application Publication
(10) Pub. No.: US 2018/0154028 A1
(43) Pub. Date: Jun. 7, 2018

(54) BAGS FOR SANITIZING CONTENTS USING OZONE AND ULTRAVIOLET LIGHT

(71) Applicant: Trizone, LLC, Santa Ana, CA (US)

(72) Inventors: Yong Tim Offutt, Los Angeles, CA (US); Samuel R. Edwards, JR., Los Angeles, CA (US)

(21) Appl. No.: 15/835,283

(22) Filed: Dec. 7, 2017

Related U.S. Application Data
(60) Provisional application No. 62/431,396, filed on Dec. 7, 2016.

(51) Int. Cl. A61L 2/10; A61L 2/20; A61L 2/24

(52) U.S. Cl. CPC A61L 2/10; A61L 2/202; A61L 2202/26; A61L 2202/14; A61L 2202/16; A61L 2/24

(57) ABSTRACT

A portable bag may comprise a body having one or more compartments each sized to receive an article of clothing. The bag may include an access channel that is configured to allow wired access to a first enclosure from a power supply external to the body. The bag may include a sanitization device comprising a hardware processor, a UV light emitter, an ozone generator, a ventilator, and a battery.

### CROSS REFERENCE TO RELATED APPLICATIONS

This application claims the benefit of U.S. Provisional Application No. 62/431,396, filed Dec. 7, 2016, the entire contents of which are hereby incorporated by reference for all that they contain and are made part of this specification.

### BACKGROUND

### Field

Embodiments of the systems and methods described herein relate to deodorizing and/or sanitizing contents of bags using ozone and/or ultraviolet light.

### Description of the Related Art

Although some related products exist, there is a need for improved systems and methods for using bags to deodorize and/or sanitize contents of a bag using ozone and/or ultraviolet light.

### SUMMARY

The systems, methods, and devices described herein each have several aspects, no single one of which is solely responsible for its desirable attributes. Without limiting the scope of this disclosure, several non-limiting features will now be described briefly.

The smart system can use ozone and/or UV light to deodorize and/or sanitize, which can discharge into the bag at the direction of a user (e.g., by touch of a button). The portable system may generate enough power to run multiple cycles. It can also be equipped with a power bank feature that charges a user's mobile device. The system may optionally be controlled and accessed through a mobile app.

Additional embodiments of the disclosure are described below in reference to the appended claims, which may serve as an additional summary of the disclosure.

In various embodiments, computer-implemented methods are disclosed in which, under control of one or more hardware computing devices configured with specific computer executable instructions, one or more aspects of the above-described embodiments (including one or more aspects of the appended claims) are implemented and/or performed.

In various embodiments, non-transitory computer-readable storage mediums storing software instructions are disclosed, wherein, in response to execution by a computing system having one or more hardware processors, the software instructions configure the computing system to perform operations comprising one or more aspects of the above-described embodiments (including one or more aspects of the appended claims).

Further, as described herein, various embodiments of the system may be configured and/or designed to generate user interface data useable for rendering the various interactive user interfaces described. The user interface data may be used by the system, and/or another computer system, device, and/or software program (for example, a browser program), to render the interactive user interfaces. The interactive user interfaces may be displayed on, for example, electronic displays (including, for example, touch-enabled displays).

### BRIEF DESCRIPTION OF THE DRAWINGS

FIG. 1 is a top front view of the mobile device with app, an and an open view of the bag according to one embodiment.

FIG. 2 is a front view of an ozone generator with an on/off control button, battery level light indicators, USB port, Micro USB port, and USB port on the side according to one embodiment.

FIG. 3A illustrates a perspective view of an example bag with available compartments accessible by zipper, including an access port to a sanitization device, and a shoulder strap according to one embodiment.

FIG. 3B is a view of the sanitization device of FIG. 1 showing the open view of an example bag with side zipper pockets, top main zippers to open the bag, and shoe compartment, side handles, removable waterproof toiletry compartment, and a laptop compartment according to one embodiment.

FIG. 4A schematically shows some internal components of an example sanitization device including a fan/blower, an ozone generator, UV lights, with a processor that includes storage, a battery, Bluetooth capability, and WiFi capability according to one embodiment.

FIG. 4B schematically shows some internal components of an example sanitization device that includes one or more sensor(s) according to one embodiment.

FIG. 4C schematically shows some internal components of an example sanitization device that includes a second fan/blower according to one embodiment.

FIG. 5A is a view showing on line 104 of FIG. 1 showing the dashboard of the mobile app with start/stop slider, name/profile, battery status, time duration according to one embodiment.

FIG. 5B shows a page of a mobile app that controls the ozone generator by using various menu options and/or syncing options according to one embodiment.

FIG. 5C shows another page of the mobile app. according to one embodiment.

FIG. 6 shows another page of the mobile app illustrating functionality of a GPS location map according to one embodiment.

FIG. 7A shows an ability to schedule events on the mobile app including a calendar to set time, date and duration for a user's scheduled cycle(s) according to one embodiment.

FIG. 7B shows a view of a completed scheduling according to one embodiment.

FIG. 7C shows the completion of system on the mobile app with a push notification, displaying “You're Fresh” according to one embodiment.

Although certain embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### DETAILED DESCRIPTION

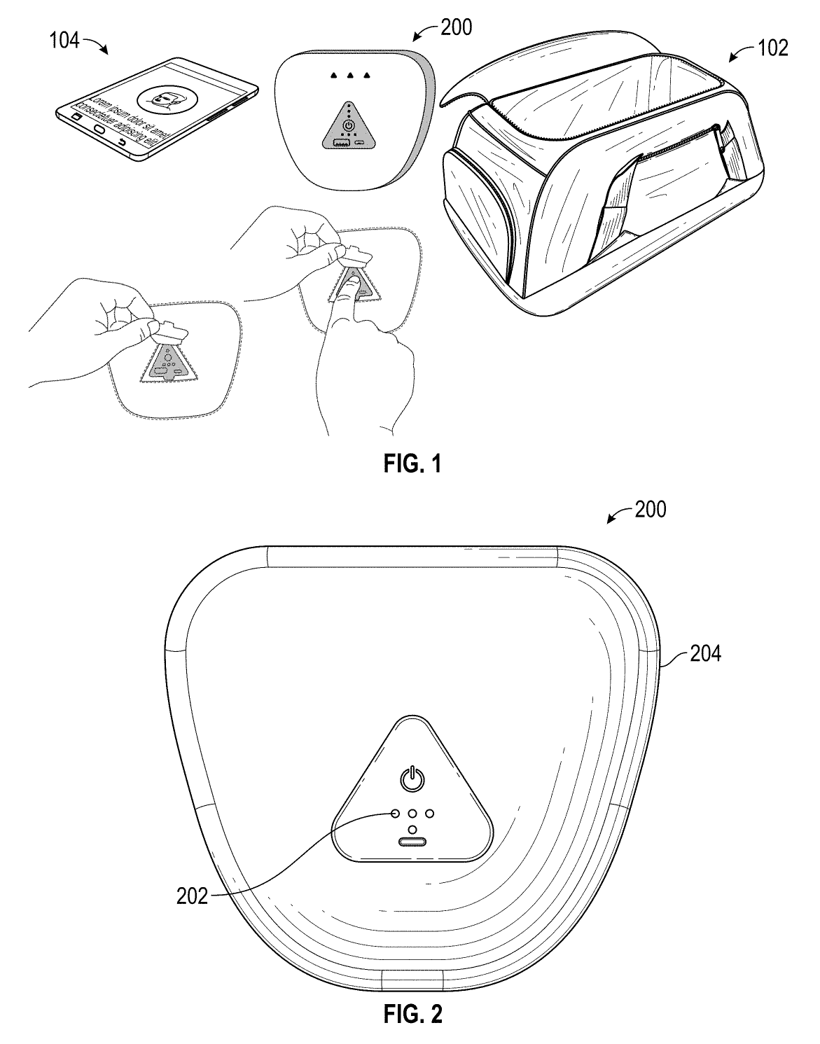

FIG. 1 illustrates a bag 102, which houses a sanitization device (not shown) in communication with a mobile device 104. As discussed in further detail below, the sanitization device advantageously provides sanitization of the bag 102, as well as one or more objects that are within the bag, such as clothing, sports equipment, etc. In the example of FIG. 1, the mobile device 104 executes a software application (such as an application that is downloadable from an application store) that communicates with the sanitization device to coordinate sanitization of the bag 102. Depending on the embodiment, the bag may comprise a duffel bag, gym bag, travel bag, suitcase, sports equipment bag, locker bag, or any other type of portable container that may benefit from periodic sanitization. For purposes of this disclosure, the sanitization device is discussed with reference to a bag or a duffel bag; however, such references are for purposes of illustration only and any references to a bag or a duffel bag should be interpreted as equally applicable to any other type of bag or enclosure.

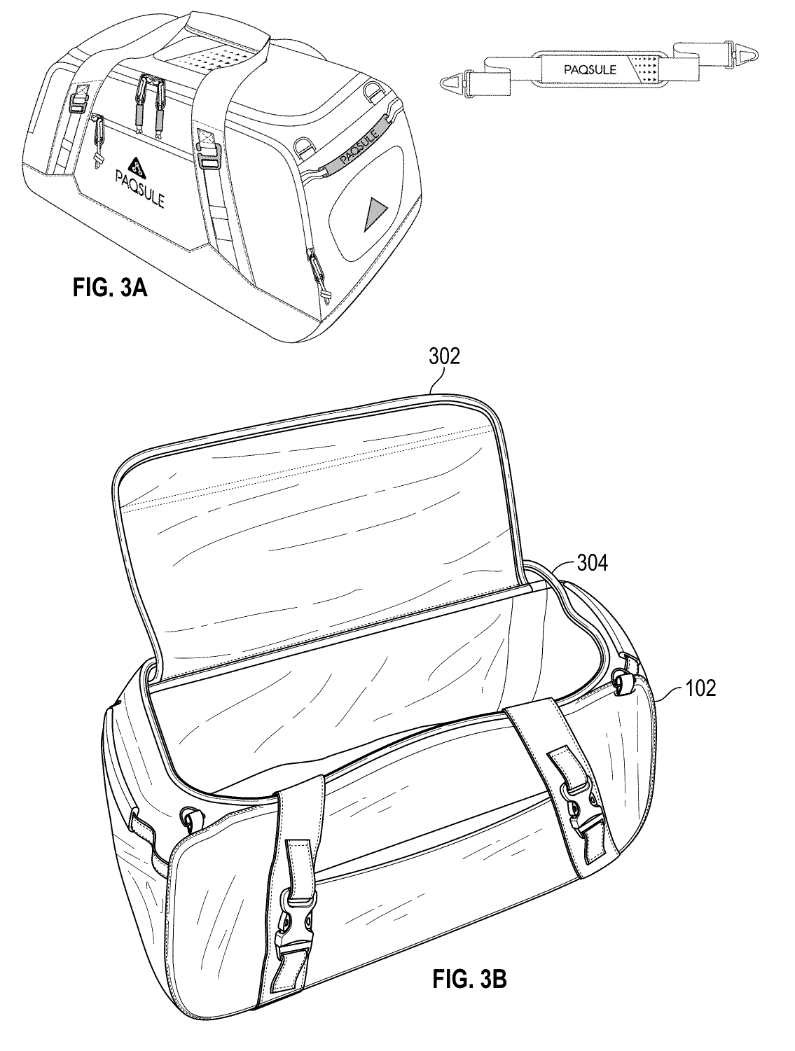

FIG. 2 illustrates an example sanitization device 200 that may be configured to provide sanitization in a bag, such as a duffel bag. In this embodiment, the sanitization device 200 includes one or more status lights 202 and a power button 204. The sanitization device 200 may include a USB port on a front side, a Micro USB port, and/or a USB port on the side of the sanitization device 200. Integrated LED lights may be installed in the housing of the sanitization device 200. The status lights 202 indicate a power level of the device (e.g., a level of the battery of the device) and/or the current sanitization activity level (e.g. from no sanitization to a full power sanitization currently occurring). The power button 204 may enable a user to manually turn the sanitization device 200 on and off. In some embodiments, the sanitization device 200 is turned on and off via a remote app, such as executing on the mobile device 104 (FIG. 1). In some embodiments, the sanitization device 200 may not include the power button 204 and/or the status lights 202, such as in an embodiment wherein the sanitization device 200 is not visible to the user and remote operation of the sanitization device 200 is preferred. FIG. 3 illustrates the bag 102 with a top 302 opened, such as unzipped in this embodiment. In some embodiments, one or more inner surfaces 304 of the bag 102 may be made of, or covered with, a material that increases sanitization effectiveness within the bag 102. For example, the inner surfaces 304 may include a material that is highly reflective of UV wavelengths of light, which are used by the sanitization device 200. In some embodiments, one or more of the inner surfaces 304 may comprise a material that allows ozone to better penetrate the material, such that sanitization of those inner surfaces 304 is optimized.

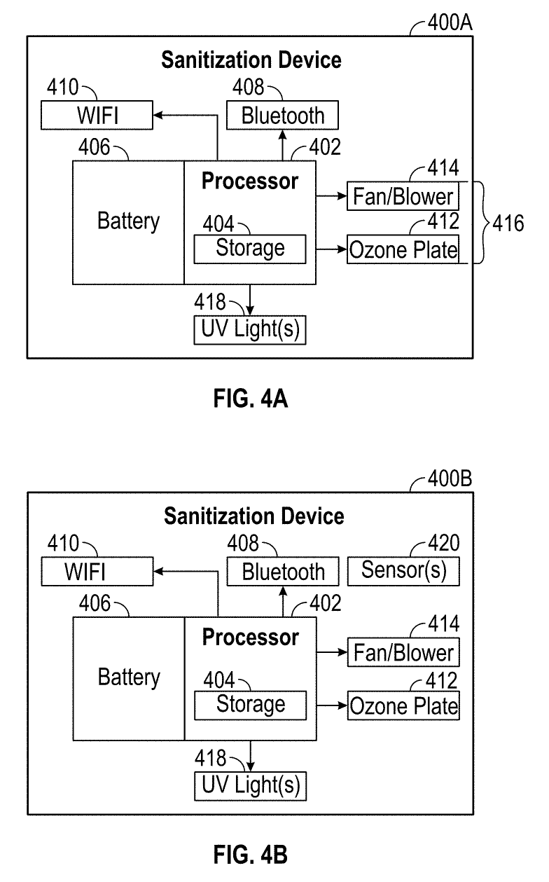

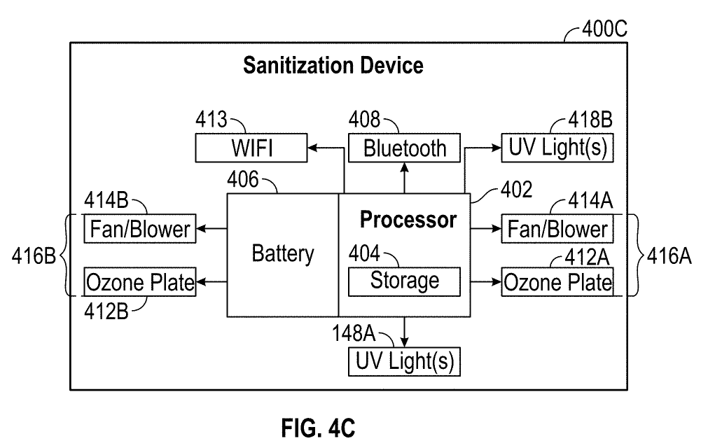

FIGS. 4A, 4B, and 4C are block diagrams illustrating exemplary components of sanitization devices 400 (including 400A, 400B, and 400C). Depending on the embodiment, sanitization devices may include fewer or additional components—the examples shown are illustrative of certain embodiments of sanitization devices.

Beginning with FIG. 4A, the sanitization device 400A includes a processor 402 in communication with storage 404 and a battery 406. While the battery 406 is shown as a single component in FIG. 4A, in some embodiments multiple batteries 406 may be included in a sanitization device, such as a first battery that provides power to the processor 402, and a second battery that provides power to the sanitization components (e.g., the ozone sanitizer 416 and/or the UV lights 418). In some embodiments, the sanitization device 400 may include one or more charging ports for external devices, such as one or more USB ports that allow charging of a mobile device external to the bag by connecting a power port of the mobile device to the USB port on the sanitization device 400. The bag in which the sanitization device 400 is placed may include a charging interface in electrical communication with the battery, wherein the charging interface is configured to receive power from a power supply external to a body of the bag. The bag may include an access channel through which wired access to a first enclosure from a power supply external to the body may be provided. In some designs, the access channel is sized for electrical wires to pass therethrough. For example, the access channel may have a dimension (e.g., diameter, width) of less than 2 cm and may be less than 1 cm.

Depending on the embodiment, the processor 402 may include various microprocessors that may be configured to control operations of the sanitization device 400A. The storage 404 includes a non-transitory storage medium, such as a solid state storage device, that may store software instructions executable on the processor 402 to operate the sanitization device 400A. Additionally, the storage 404 may store user preferences, such as preferences for sanitizing a bag, as well as logs of previous sanitization processes that have been performed, and some embodiments historical data from sensors within the bag (such as that monitor moisture or odor level within the bag).

The processor 402, executing the software instructions, interfaces with various modules and/or components, such as via a communication bus and/or separate communication connections to the components. Advantageously, the processor 402 coordinates a sanitization process within a bag in which the sanitization device is placed (such as at the time of fabricating the bag or post fabrication when the sanitization device is placed in a preconfigured pocket, pouch, etc. configured to hold the sanitization device and to provide access for the device within the bag). The sanitization process may vary depending on the embodiment. For example, the user may establish a sanitization process (e.g. via the user device 104) that automatically provides a particular time period of sanitization within the bag on a periodic basis. For example, the sanitization process may include activation of both and ozone sanitizer 416 and UV lights 418 for 15 minutes every 24 hours (e.g., each morning after a user has visited a gym and placed soiled clothing in the bag). In some embodiments, other time periods (e.g., 5 minutes, 10 minutes, 15 minute, 20 minutes, 25 minutes, 35 minutes, etc.) and periodic schedules for sanitization (e.g., every one hour, six hours, 12 hours, one week, etc.) may be implemented in response to user selection of those attributes and/or automatic determination of the sanitization process.

In some embodiments, the processor 402 causes storage of log data regarding operations of the various components of the system. For example, data regarding sensors readings, sanitization device activation times, periods, etc., may be stored in the storage 404 and/or transmitted to an external computing device. In some embodiments, the log data may be analyzed to determine adjustments to a sanitization schedule, such as based on identifying pulsing variations of the UV and ozone devices that provides improved sanitization (e.g., based on the logged sensor data). In some embodiments, machine learning algorithms are applied to the historical log data to provide suggestions (which may be approved by use input or automatically implemented) for optimizing sanitization.

The sanitization process may include automatic activation of one or more sanitization components (e.g. the ozone sanitizer 416 and/or UV lights 418) based on feedback from one or more sensors within the bag (e.g. such as sensors 420 illustrated in sanitization device 400B), such as to automatically sanitize the bag at a certain level (e.g. for a calculated time and/or calculated sanitization level) when and odor and/or moisture level within the bag exceeds a pre-set threshold (e.g., set by the user, such as based on initial sensor values when the user's soiled clothing is placed in the gym bag initially at the time of setting up a sanitization schedule).

In the example of FIG. 4A, the processor 402 communicates with a Bluetooth transceiver 408 and/or a Wi-Fi transceiver 410 that enables communications with a mobile device, such as mobile device 104 of FIG. 1. Depending on the embodiment, the sanitization device 400 may include fewer communication transceivers and/or different communication transceivers. In some embodiments, the software application on the mobile device 104 allows the user to control which of the communication transceivers are used (e.g. powered on) for communication with the user. Accordingly, power used by the sanitization device 400 may be optimized based on those user preferences. As discussed elsewhere herein, the mobile device 104 may communicate with the sanitization device 400 (e.g. via the Wi-Fi for 10 or Bluetooth correlate transceivers) to provide a schedule for sanitization of the bag, the two passed sanitization activities, view sensor information obtained by the sanitization device 400, tracked location of the sanitization device 400 (and the bag), and other related tasks.

In the example of FIG. 4A, an ozone sanitizer 416 is illustrated in communication with the processor 402. In this embodiment, the ozone sanitizer 416 comprises an ozone plate 412 and a fan/blower 414. In some designs, the fan/blower 414 may be referred to as a ventilator. Depending on the implementation, a fan (which generally circulates air) and/or a blower (which generally pushes air) may be used in conjunction with an ozone plate 412 that generates ozone. An example blower may be a microblower, such as one with a dimension (e.g., length) of less than 5 cm (e.g., about 3 cm). An example fan may have a length of less than 3 cm (e.g., about 1.8 cm). The fan/blower 414 may be configured to rotate at between about 8,000 rpm and 13,000 rpm. In some designs, the rotation is about 11,000 rpm. In some embodiments, an ozone coil may be used in place of, or in addition to, the ozone plate 412. Additionally, in other embodiments other ozone generation components may be used.

The ozone plate 412 may be approximately a rectangular prism in shape. The ozone plate 412 may have a length that is at least twice the width. For example, the length may be between about 6 cm and 14 cm. The width may be between about 3 cm and 8 cm. The ozone plate 412 can produce a concentration of ozone in the sanitization device 400 or compartments therein sufficient to deodorize and/or sanitize objects (e.g., articles of clothing). For example, the ozone plate 412 can produce a concentration of ozone between about 0.001 ppm and 0.015 ppm. In some embodiments, the ozone plate 412 may be configured to produce a concentration of ozone of between about 0.005 ppm and 0.0075 ppm. In some designs, the concentration of ozone is between 0.01 ppm and 0.045 ppm. In some embodiments, the concentration is about 0.02 ppm.

In some designs, the ozone plate 412 is configured to produce a level of ozone below a country's maximum allowed concentration of ozone that comes in contact with humans. For example, the ozone plate 412 may be configured to produce a concentration of ozone below standards set by the United States Environmental Protection Agency (EPA), such as below 0.08 ppm.

The ozone plate 412 may be configured to output one or more rates of output depending on the size of the enclosures, the size of the bag, the size of the sanitization device 400, the types and/or concentrations of pathogens present in the bag, and/or other factors. For example, an ozone plate 412 in smaller bags may require a smaller rate, and higher concentrations of pathogens may require higher output by the ozone plate 412. The ozone plate 412 may be configured to output between about 200 mg per hour and 800 mg per hour of ozone. In some designs, the ozone plate 412 is configured to output between 350 mg of ozone per hour and 650 mg per hour. In some embodiments, the output rate is about 500 mg/h (e.g., for equipment bags). The sanitization device 200 may include a tubal delivery system comprising one or more tubes configured to promote the delivery of ozone throughout the bag. For example, a first end of a tube in the tubal delivery system may be disposed adjacent or near the ozone plate 412. A second end of the tube may be disposed in an enclosure within the bag, such as an adjacent enclosure from the location of the ozone plate 412.

Without being limited by theory, it is believed that ozone is capable of passing (e.g., diffusing) through a protein layer of a virus or bacteria (or other microorganism) and into its core, which contains nucleic acids, and disrupting and/or destroying one or more of the nucleic acids. In some cases, the ozone destroys the capsid of the virus by oxidation. In bacteria and other pathogens, the ozone ruptures the cell wall. With the reduction of bacteria or other pathogens that produce smells or odors, the odor is similarly reduced or eliminated. It is also believed that ozone can neutralize and/or deodorize inorganic toxins, particulate matter, bacteria, and/or airborne resins (e.g., cigarette smoke), and/or other particles.

In the example of FIG. 4A, one or more Ultraviolet lights 418 are also in communication with the processor 402. In some embodiments, the UV lights 418 include a strip of lights (e.g., LED lights) that are positioned on an inner surface of the bag so that coverage of UV light emitted by the lights 418 within the bag is optimized. In some designs, the UV light(s) 418 include or one or more UV bulbs (e.g., connected to a housing of the sanitization device 400). The UV light(s) 418 may include 2, 3, 4, 6, or more bulbs and/or LEDs.

The UV light(s) 418 can output wavelengths of light sufficient to neutralize and/or kill pathogens or other microorganisms (e.g., bacteria, viruses, fungi). The wavelengths may include those in the UV-C band. The UV light(s) 418 may be configured to output wavelengths in any range within the range of 100 nm and 280 nm. For example, the UV light(s) 418 may be configured to emit light between about 230 and 310 nm. In some designs, the UV light(s) 418 is/are configured to emit light between 260 nm and 290 nm. Without being limited by theory, it is believed that light between 260 nm and 290 nm beneficially balances safety to humans while providing high effectiveness in sanitization of certain pathogens. In some designs, light at about 280 is used. Other ranges or wavelengths, however, are possible.

The use of UV lights and ozone may be provided within the bag during specific sessions or cycles. Each cycle may last a predetermined amount of time. For example, each cycle may last 3 minutes, 5 minutes, 10 minutes, 15 minutes, 20 minutes, 25 minutes, 30 minutes, 35 minutes, or 45 minutes depending on the size of the bag and the intensity of the UV light(s) 418 and ozone plate 412 used. In some designs, one or more sensor(s) 420 are provided within the bag to detect a level of microorganisms within the bag. In such designs, each cycle may be determined based on the level of readings in the bag. The sensor may be in communication with one or more of the processor 402, storage 404, and/or battery 406. In some designs, the sensor may be in direct communication with the UV light(s) 418 and/or ozone sanitizer 416. Each cycle may be spaced from adjacent cycles by a waiting period. The waiting period may be a few seconds or a few minutes. For example, the waiting period may be 1 minute, 2 minutes, 3 minutes, 5 minutes, 10 minutes, 15 minutes, or 20 minutes. The waiting period may be based on the size of the battery used, the intensity and/or number of UV light(s) 418, and/or the rate of the ozone plate 412. In some designs, in order to increase germicidal effectiveness of the UV light(s) 418, the UV light(s) 418 may be operated during cycles in which the fan/blower 414 is in operation. In this way, the fan/blower 414 can move the microorganisms through the rays emitted by the UV light(s) 418, thus increasing the effectiveness of the sanitization device 400.

The one or more sensor(s) 420 may include one or more of an olfactometer (e.g., electric nose), a chemosensor, a moisture meter, an optical sensor, a motion sensor, and/or a proximity sensor. It may be beneficial to use one or more sensors to improve the functioning of the bag. For example, a bag may comprise an olfactometer, which may include a chemosensor, to detect a level of odor within the bag. Additionally or alternatively, the bag may comprise a moisture meter to detect a level of moisture (e.g., humidity) in the bag or part of the bag (e.g., a bag compartment). The olfactometer and/or moisture meter may be configured to send a signal to the processor 402. The processor 402 may, in response to the signal, cause the ozone plate 412 and/or the UV light(s) 418 to turn on and begin emission. In this way, the bag may be outfitted with an automatic means for deodorizing and/or sanitizing the inside of the bag.

It may be further advantageous to automatically shut off the functioning of the ozone plate 412 and/or ozone sanitizer 416. The bag may comprise one or more motion sensors and/or proximity sensors in order to detect whether the bag (or compartment within the bag) will cause a user to come in contact with a threshold level of ozone and/or UV light. For example, the motion sensor may detect unzipping of the bag's compartment and may send a signal to the processor 402 to shut off the ozone plate 412 and/or UV light(s) 418. Additionally or alternatively, the proximity sensor may be configured to determine that a user is within a threshold distance and may, in response to this threshold distance, be configured to send a signal to the processor 402, which may cause the ozone plate 412 and/or UV light(s) 418 to shut off, such as to prevent the user from interacting directly with ozone and reducing possible skin irritation that may be caused by ozone. In some designs, the motion sensor and/or proximity sensor may be configured to reengage the ozone plate 412 and/or UV light(s) 418, such as by sending a signal to the processor 402 to reengage the ozone plate 412 and/or UV light(s) 418 once the motion sensor no longer detects motion (which may indicating that users are no longer near the bag). The proximity sensor may be similarly configured. The motion sensor and/or proximity sensor may comprise one or more optical, microwave, and/or acoustic sensors, either passive or active.

In some designs, the battery 406 may comprise one or more lithium ion and/or lithium polymer batteries. The battery 406 may be approximately a rectangular shape with a length of between about 45 mm and 85 mm, or any other shape or size suitable for placement in a compartment of a bag. In some designs, the length is about 65 mm. The battery 406 may have a width of between about 45 mm and 85 mm. In some designs, the battery 406 has a width of about 18 mm. The battery 406 can be configured to provide enough power to provide one to three full charges of a mobile device (e.g., smartphone). In some embodiments, the battery 406 can provide two full charges of the mobile device. Additionally, or alternatively, the battery 406 may be configured to provide at least three 35-minute cycles of ozone and/or UV lights.

In the examples of FIGS. 4B-4C, the example sanitization devices 400B and 400C each include certain components similar to those discussed with reference to sanitization device 400A, as indicated by the similar numbering of those components. Additionally, the sanitization device 400B includes one or more sensors 420, such as moisture, odor, and/or optical sensors that may provide input to the processor 402 in order to track effectiveness of the sanitization device and/or provide inputs to automate certain operations of the sanitization device. For example, in some embodiments the sanitization device 400 can automatically provide sanitization to a bag, such as by turning on the UV lights 418 and ozone sanitizer 416, when and odor level detected at the sensor reaches a predetermined level. In some embodiments, sensors may be located at various locations within the bag, such as on a lid, ends of the bag, and/or bottom of the bag, for example. In some embodiments, a sensor may be placed on the outside of the bag, such as to provide a comparison of ambient odors or moisture with the sensed internal moisture and odor from sensors within the bag.

The bag may contain one or more compartments in which items (e.g., articles of clothing) may be placed. Each compartment may be sized to adequately fit specific items. For example, one compartment may be sized to fit a pair of shoes while another may be differently sized to fit particular athletic gear (e.g., football pads, shin guards, helmet, racket, mask, etc.). One or more of the compartments may be associated with corresponding enclosures. In some designs, each enclosure is adapted to fit a sanitization device 400 or one or more individual elements of a sanitization device 400. For example, an enclosure may be sized to fit one or more UV light(s) 418 and/or ozone sanitizer 416 (or just the ozone plate 412). In order to provide electrical communication between/among two or more enclosures, the bag may comprise one or more channels through which electrical communication devices (e.g., wires, LED strips) may be passed. In some embodiments, communications between a microprocessor and multiple sanitization devices in separate enclosures of a bag may be achieved via wireless communication, such as Bluetooth or other short-range wireless communication protocol. The bag may have a mass of between 0.20 kg and 0.85 kg. Other masses or weights are possible.

In the example of FIG. 4C, the sanitization device 400C includes multiple ozone sanitizers 416A and 416B, as well as multiple UV light sources 418A and 418B. Fewer or additional sanitizers and/or 418 may be used, as well as one or more other types of sanitizers. In some designs (not shown), the ozone sanitizer 416A may be in communication (e.g., wireless communication) with the ozone sanitizer 416B. In this way, the processor 402 may be configured to communicate directly with only one of the ozone sanitizer 416A and indirectly with the ozone sanitizer 416B. The UV lights 418A may be placed on one end of a bag with UV lights 418B placed on an opposite end of the bag (or top and bottom of the bag alternatively). Similarly, ozone sanitizer 416A may be placed on one end of a bag with ozone sanitizer 416B placed on the other end of the bag (or top and bottom of the bag alternatively). In one embodiment, the battery 406, processor 402, storage 404 or adjacent to the ozone sanitizer 416, while electrical connections (or other communication channel) extends along at least one side of the bag to where sanitization scratch that ozone sanitizer 416B is positioned. The multiple ozone sanitizers 416 and/or UV sources 418 may operate concurrently based on a common control signal from the processor 402 (e.g., to each of the ozone sanitizers 416) or maybe separately controlled in some embodiments by the processor 402 (e.g., the UV light 418A may be operated at different times, frequencies, periods, then UV lights 418B). The processor 402 may be configured to send data from the one or more sensor(s) 420 to the storage 404. The storage 404 may be configured to track data from the one or more sensor(s) 420. The data may be stored for a week, a month, a year, or longer.

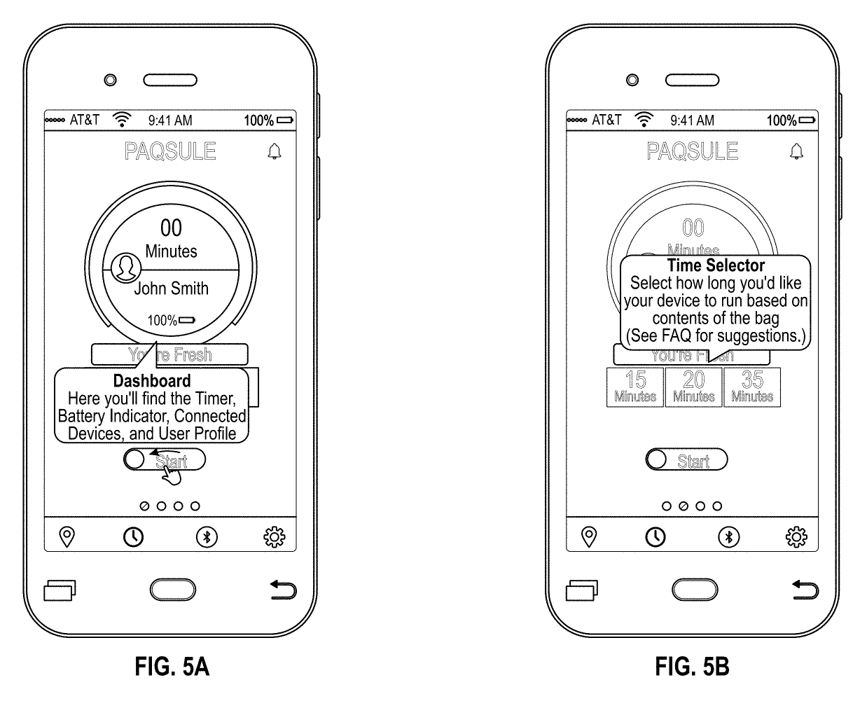

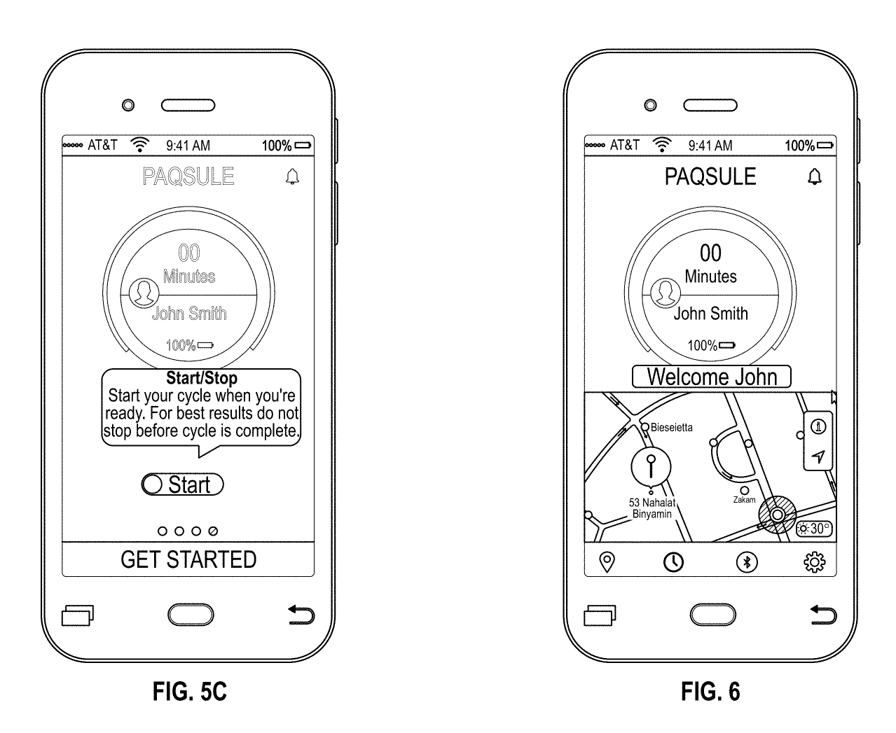

FIGS. 5A, 5B, and 5C are example screenshots from a mobile device, such as user device 104 of FIG. 1, such as may be presented by an application executing on the mobile device that is in communication with a sanitization device 200 or 400. In the examples of FIG. 5, a user is provided with options for setting a timer for sterilization of a bag, wherein sterilization may be performed by one or more sterilization components, such as an ozone sterilizer and/or UV sterilizer.

FIG. 6 illustrates an example of a sanitization mobile app that displays a location of a sanitization device, and correspondingly the bag within which the sanitization devices placed. In this example, the sanitization device includes geolocation circuitry, such as a GPS transceiver and/or is configured to use Wi-Fi location information to determine a location of the sanitization device and allow the sanitization application to generate a map user interface indicating that location.

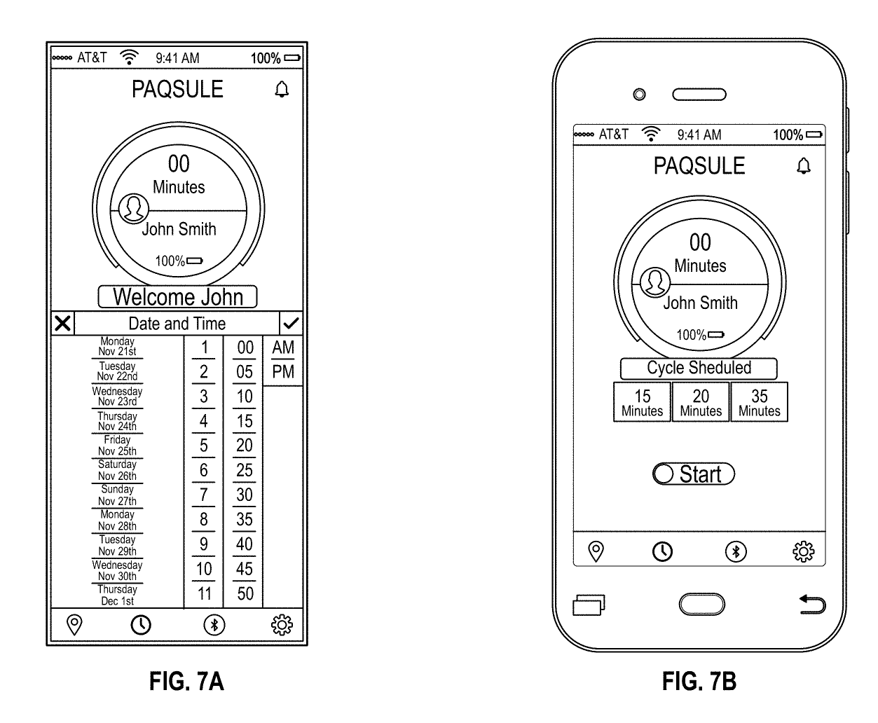

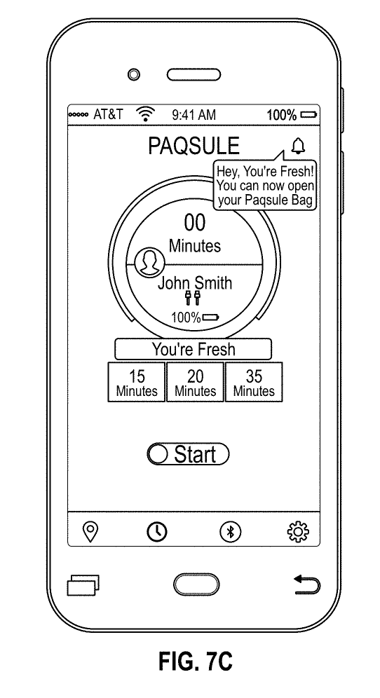

FIGS. 7A-7C illustrate various views within a mobile app, including a timer of sanitization processes that a user can set for a particular sanitization device. In this example, the user may select a date and time for the next cycle for the sanitization device 400. The user may be able to select a number of cycles in each day, a length of one or more of the cycles, and/or a length of one or more of the waiting periods between cycles. In some designs, the user may select a threshold level of sanitization above which the sanitization device 400 automatically begins a sanitization cycle.

Depending on the embodiment, a sanitization bag, which may include one or more sanitization device, may include any combination of the components and features below, as well as any combination of any other components and features discussed herein:

Ozone Generator that provides from 200 mg-500 mg

Integrated UV lights

Fan/Blower

On/Off button

USB Charging Bank

USB-C Connector

Power source access and Battery

Rechargeable lithium polymer battery

Charging via USB to computer system or power adapter

Splash, Water, and Dust Resistant

Rated IP67 under IEC standard 60529

Wireless

BLE Bluetooth

App Controls

Multiple UV and/or ozone sanitizers (e.g., Dual Fresh System)

Clone Device

Display

LCD—Timer—Battery life—Odormeter level, Sweatometer levels

Touch Screen

Fingerprint-resistant oleophobic coating

Voice Control

Use your voice to turn on and off

Get intelligence on time of completion, odor and sweat levels

Activate with only your voice using “PaqTech”

Sensors

Odormeter

Sweatometer

Proximity sensor

### Example Embodiments

In one embodiment a sanitization device comprises a body having one or more compartments each sized to receive one or more articles of clothing, one or more enclosures, each of the one or more enclosures disposed adjacent a corresponding compartment, and an access channel configured to allow temporary wired access to a first enclosure from a power source external to the body

In some embodiments, the device comprises a sanitization device having a light emitter configured to emit light into the one or more compartments, the light having a wavelength between about 260 nm and 290 nm, an ozone generator configured to release ozone into the one or more compartments at a rate of between about 350 mg per hour and 650 mg per hour, a ventilator configured to circulate ozone from the ozone generator throughout the one or more compartments, a hardware processor configured to selectively activate the light emitter and the ozone generator in accordance with a sanitization schedule, a battery configured to provide electrical power to the sanitization device for at least ten minutes, the battery disposed within the first enclosure, and a charging interface in electrical communication with the battery, the charging interface configured to receive power from the power source to recharge the battery.

In some embodiments, the sanitization device is configured to provide a first compartment adjacent the first enclosure a level of ozone concentration of between about 0.02 ppm and 0.08 ppm.

In some embodiments, the light emitter comprises one or more LEDs.

In some embodiments, the sanitization device further comprises a communication module comprising Bluetooth or WiFi communication circuitry configured to communicate wirelessly with a mobile computing device. In some embodiments, the mobile computing device executes a software application configured to generate the sanitization schedule in response to user inputs on the mobile computing device, and to communicate the sanitization schedule to the sanitization device.

In some embodiments, the sanitization schedule is dynamically adjusted in response to sensor data from one or more sensors positioned within the bag.

In some embodiments, the processor is configured to automatically activate one or more of the light emitter and ozone generator in response to an output level from a sensor positioned within the bag reaching a predetermined level and to deactivate the one or more of the light emitter and ozone generator in response to the output level from the sensor positioned within the bag dropping below a second predetermined level. In some embodiments, the sanitization device communicates wirelessly with a mobile computing device executing a sanitization software application configured to receive user input indicating one or more of the first and second predetermined levels, and communicating those levels to the processor.

In some embodiments, the device further comprise one or more storage devices configured to store software code to selectively activate the light emitter and ozone generator. In some embodiments, the one or more storage devices is further configured to store log data indicating operations of components of the bag, including the light emitter and ozone generator, as well as any sensors within the bag. In some embodiments, wherein the processor or a remote computing system is configured to access the log data and determine updates to the sanitization schedule based on one or more patterns detected in the log data.

In some embodiments, the body has a mass of between 0.20 kg and 0.85 kg.

In some embodiments, the device further comprises one or more chemical sensor, moisture sensor, or optical sensor.

In some embodiments, the device further comprises an optical sensor configured to detect a threshold event and, based on the threshold event, to automatically send a signal to the processor, the processor configured to disengage the light emitter. In some embodiments, the threshold event comprises an opening of the bag.

In some embodiments, the device further comprises a USB charge port coupled to the battery, wherein the battery stores charge sufficient to recharge a mobile device on a single charge.

In some embodiments, the ventilator has a length of less than 5 cm.

In some embodiments, the device further comprises a second sanitization device comprising a second ventilator and a second ozone generator.

In some embodiments, the sanitization device is disposed primarily within the first enclosure.

It is contemplated that the particular features, structures, or characteristics of any embodiments discussed herein can be combined in any suitable manner in one or more separate embodiments not expressly illustrated or described. In many cases, structures that are described or illustrated as unitary or contiguous can be separated while still performing the function(s) of the unitary structure. In many instances, structures that are described or illustrated as separate can be joined or combined while still performing the function(s) of the separated structures.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Thus, it is intended that the scope of the inventions herein disclosed should not be limited by the particular embodiments described above, but should be determined by a fair reading of the claims that follow.

Any process descriptions, elements, or blocks in the flow diagrams described herein and/or depicted in the attached figures should be understood as potentially representing modules, segments, or portions of code which include one or more executable instructions (as described below) for implementing specific logical functions or steps in the process. Alternate implementations are included within the scope of the embodiments described herein in which elements or functions may be deleted, executed out of order from that shown or discussed, including substantially concurrently (for example, through multi-threaded processing, interrupt processing, or multiple processors or processor cores or on other parallel architectures) or in reverse order, depending on the functionality involved.

Any of the methods and processes described above may be partially or fully embodied in, and partially or fully automated via, logic instructions, software code instructions, and/or software code modules executed by one or more general purpose processors and/or application-specific processors (also referred to as “computer devices,” “computing devices,” “hardware computing devices,” “hardware processors,” and the like). For example, the methods described herein may be performed as software instructions are executed by, and/or in response to software instruction being executed by, one or more hardware processors (e.g., one or more processors of the computing system 150) and/or any other suitable computing devices. The software instructions and/or other executable code may be read from a tangible computer-readable medium. A tangible computer-readable medium is a data storage device that can store data that is readable by a computer system and/or computing devices. Examples of computer-readable mediums include read-only memory (ROM), random-access memory (RAM), other volatile or non-volatile memory devices, DVD-ROMs, CD-ROMs, magnetic tape, flash drives, and/or optical data storage devices. Accordingly, a software module may reside in RAM memory, flash memory, ROM memory, EPROM memory, EEPROM memory, registers, hard disk, solid state drive, a removable disk, a CD-ROM, a DVD-ROM, and/or any other form of a tangible computer-readable storage medium.

Additionally, any of the methods and processes described above may be partially or fully embodied in, and partially or fully automated via, electronic hardware (for example, logic circuits, hardware processors, and/or the like). For example, the various illustrative logical blocks, methods, routines, and the like described in connection with the embodiments disclosed herein may be implemented as electronic hardware, computer software, or combinations of both. To illustrate this, various illustrative components, blocks, modules, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the disclosure.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain embodiments require at least one of X, at least one of Y, and at least one of Z to each be present.

While the above detailed description has shown, described, and pointed out novel features as applied to various embodiments, it may be understood that various omissions, substitutions, and changes in the form and details of the devices or processes illustrated may be made without departing from the spirit of the disclosure. As may be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A portable bag comprising:
a body comprising:
one or more compartments each sized to receive one or more articles of clothing;
one or more enclosures, each of the one or more enclosures disposed adjacent a corresponding compartment;
an access channel configured to allow temporary wired access to a first enclosure from a power source external to the body;
a sanitization device comprising:
a light emitter configured to emit light into the one or more compartments, the light having a wavelength between about 260 nm and 290 nm;
an ozone generator configured to release ozone into the one or more compartments at a rate of between about 350 mg per hour and 650 mg per hour;
a ventilator configured to circulate ozone from the ozone generator throughout the one or more compartments;
a hardware processor configured to selectively activate the light emitter and the ozone generator in accordance with a sanitization schedule;
a battery configured to provide electrical power to the sanitization device for at least ten minutes, the battery disposed within the first enclosure; and
a charging interface in electrical communication with the battery, the charging interface configured to receive power from the power source to recharge the battery.

2. The portable bag of claim 1, wherein the sanitization device is configured to provide a first compartment adjacent the first enclosure a level of ozone concentration of between about 0.02 ppm and 0.08 ppm.

3. The portable bag of claim 1, wherein the light emitter comprises one or more LEDs.

4. The portable bag of claim 1, the sanitization device further comprising:
a communication module comprising Bluetooth or WiFi communication circuitry configured to communicate wirelessly with a mobile computing device.

5. The portable bag of claim 4, wherein the mobile computing device executes a software application configured to generate the sanitization schedule in response to user inputs on the mobile computing device, and to communicate the sanitization schedule to the sanitization device.

6. The portable bag of claim 1, wherein the sanitization schedule is dynamically adjusted in response to sensor data from one or more sensors positioned within the bag.

7. The portable bag of claim 1, wherein the processor is configured to automatically activate one or more of the light emitter and ozone generator in response to an output level from a sensor positioned within the bag reaching a predetermined level and to deactivate the one or more of the light emitter and ozone generator in response to the output level from the sensor positioned within the bag dropping below a second predetermined level.

8. The portable bag of claim 7, wherein the sanitization device communicates wirelessly with a mobile computing device executing a sanitization software application is configured to receive user input indicating one or more of the first and second predetermined levels, and communicating those levels to the processor.

9. The portable bag of claim 1, further comprising:
one or more storage devices configured to store software code to selectively activate the light emitter and ozone generator.

10. The portable bag of claim 9, wherein the one or more storage devices is further configured to store log data indicating operations of components of the bag, including the light emitter and ozone generator, as well as any sensors within the bag.

11. The portable bag of claim 10, wherein the processor or a remote computing system is configured to access the log data and determine updates to the sanitization schedule based on one or more patterns detected in the log data.

12. The portable bag of claim 1, wherein the body has a mass of between 0.20 kg and 0.85 kg.

13. The portable bag of claim 1, further comprising one or more chemical sensors, moisture sensors, or optical sensors.

14. The portable bag of claim 1, further comprising an optical sensor configured to detect a threshold event and, based on the threshold event, to automatically send a signal to the processor, the processor configured to disengage the light emitter.

15. The portable bag of claim 14, wherein the threshold event comprises an opening of the bag.

16. The portable bag of claim 1, further comprising a USB charge port coupled to the battery, wherein the battery stores charge sufficient to recharge a mobile device on a single charge.

17. The portable bag of claim 1, wherein the ventilator has a length of less than 5 cm.

18. The portable bag of claim 1, further comprising a second sanitization device comprising a second ventilator and a second ozone generator.

19. The portable bag of claim 1, wherein the sanitization device is disposed primarily within the first enclosure.
